(19) United States
(12) Patent Application Publication
(10) Pub. No.: US 2014/0324182 A1
(43) Pub. Date: Oct. 30, 2014

(54) CONTROL SYSTEM, METHOD AND COMPUTER PROGRAM FOR POSITIONING AN ENDOPROSTHESIS

(71) Applicant: SIEMENS AKTIENGESELLSCHAFT, Muenchen (DE)

(72) Inventors: RAINER GRAUMANN, HOECHSTADT (DE); GERHARD KLEINSZIG, FORCHHEIM (DE)

(73) Assignee: SIEMENS AKTIENGESELLSCHAFT, Muenchen (DE)

(21) Appl. No.: 14/260,479

(22) Filed: Apr. 24, 2014

(30) Foreign Application Priority Data
Apr. 24, 2013 (DE) 102013207463.8

(51) Int. Cl. A61F 2/46; A61F 2/32

(52) U.S. Cl. CPC A61F 2/4607; A61F 2/32; A61F 2002/4633
USPC 623/22.12

(57) ABSTRACT

A computer-aided control system, a computer-based method and a computer program or a computer program product control an implant positioning of an endoprosthesis. Planning data are generated on the basis of a preoperatively acquired first image data set. The endoprosthesis is provisionally positioned intraoperatively. A second image data set representing the current ACTUAL position of the endoprosthesis is thereupon acquired. Control data are thereupon generated, the control data serve for the fine positioning of the endoprosthesis and being based on a comparison between DESIRED and ACTUAL states.

### CROSS-REFERENCE TO RELATED APPLICATION

This application claims the priority, under 35 U.S.C. §119, of German application DE 10 2013 207 463.8, filed Apr. 24, 2013; the prior application is herewith incorporated by reference in its entirety.

### BACKGROUND OF THE INVENTION

### Field of the Invention

The present invention relates to a method, a system and a product for controlling a process of inserting endoprostheses in the context of a clinical intervention.

Owing to the increase in degenerative joint diseases (for instance as a result of arthrotic or other pathological changes), implantology and joint endoprosthetics have developed into important areas within medical technology. The term “endoprostheses” denotes implants which remain permanently in the body, for example in the form of an artificial hip joint, knee joint, shoulder joint, etc. However, use is made not only of orthopedic prostheses, but also heart valve replacement and vascular replacement prostheses or breast implants. Furthermore, dental implants are known in the field of dental technology.

In order that the service lives of the implanted endoprostheses can be maximized as much as possible, nowadays recourse is often had to measures for improving the planning and performance of an endoprosthesis implantation. Imaging methods are frequently used in this case, for example on the basis of x-ray images.

For optimum placement and positioning of a hip endoprosthesis (shaft with socket), hitherto in the prior art recourse has generally been had to pre- and intraoperatively obtained two-dimensional fluoroscopic recordings in order to plan the implantation. Fluoroscopy is likewise based on x-ray technology in which the human body (for example the hip) is radiographed by the x-ray beam and the result is displayed on a fluorescent screen. The image is subsequently processed digitally and can be displayed directly on the monitor for evaluation. In this case, it is possible to administer contrast agents (for example barium) to the patient in order to improve the visibility of specific organs.

Once the image recordings are available, the physician can then choose a suitable implant on the basis of the x-ray images and on the basis of the medical examination of the patient (for example mobility of the leg). During the insertion of the implant, however, occasionally it proves to be difficult to position the implant parts as optimally as possible in the remaining anatomical structures (for example femur, etc.). The physician generally has no further aids available during this task, and so the physician can have recourse exclusively to his knowledge based on experience.

### SUMMARY OF THE INVENTION

The present invention therefore addresses the problem of demonstrating a way of being able to improve the procedure described above. In particular, the intention is to avoid errors when planning an endoprosthesis implantation. Furthermore, the intention is for the physician to obtain in real time a possibility of visual checking which enables the physician to compare the actual position of the implants with the previous planning data with regard to correspondence. Overall, the intention is to improve the quality of the prosthesis implantation and to increase the service lives of the endoprosthesis. A further problem addressed can be seen in shortening the time for performing the implantation and providing the physician with further visual and other aids for checking quality in the prosthetics.

The solution to the problem with regard to the claimed method is described below. Features, advantages or alternative embodiments mentioned here can likewise also be applied to the other claimed subjects, and vice versa. In other words, the substantive claims (which are directed for example to a system, an apparatus or to a product) can also be developed with the features described or claimed in association with the method, and vice versa. In this case, the corresponding functional features of the method are embodied by corresponding substantive modules, in particular by hardware modules.

In accordance with one aspect, the present invention relates to a computer-implemented, image-aided method for controlling a process of inserting an endoprosthesis. The method includes providing a preoperatively acquired first image data set in a data memory. In this case, the first image data set can be acquired directly before the surgical intervention and be stored in the data memory. On the other hand it is also possible to have recourse here to an image data set already acquired, which is acquired for example in the context of a preliminary examination of the patient at an earlier time and is provided in the data memory.

Planning data is then generated for the positioning and orientation of the endoprosthesis. The planning data identify, inter alia, a DESIRED state for the endoprosthesis in the inserted state in the respective anatomical structure (such as hip, for example). Preferably, the planning data are generated on the basis of the first image data set. The endoprosthesis is provisionally inserted and a second image data set is acquired with the provisionally inserted endoprosthesis as an ACTUAL state. In this case, the ACTUAL state identifies the current location (position and orientation) of the endoprosthesis in the anatomical structure of the patient. Control data are then generated for the endoprosthesis or for the fine positioning of the endoprosthesis on the basis of an image-processing comparison between DESIRED state and ACTUAL state using a 3D/2D registration.

The concepts used in the context of this application are defined in greater detail below.

The designation “insertion” of the endoprosthesis relates to the positioning and fine adjustment of the respective implant in the anatomical structure and subsequent anchoring. The principal form of use of the present invention relates to endoprostheses for artificial hip joints. Insertion can be performed in the context of a surgical intervention. However, alternative embodiments here provide endoprostheses for other joints or else vascular implants, vascular partial implants, breast implants, etc.

The computer-implemented method is image-aided and is based on two separate recordings at least on a preoperative recording (for example 3D CT scan or 3D MRI scan) and furthermore an intraoperatively acquired image recording (for example 3D or 2D C-arc scan or a fluoro-recording). What is important is that the two different recordings (first image data set and second image data set) each identify different states for the endoprosthesis: the preoperative recording with the planning data identifies the DESIRED state of the endoprosthesis, while the second image data set with the provisionally inserted endoprosthesis represents the current ACTUAL state. By means of subsequent image processing measures, in particular using a 3D/2D registration, the first image data set and the second image data set can be analyzed with regard to correspondence or deviations. Results for the control of the endoprosthesis implantation can be determined there from and provided to the user. The control data identify, in particular, whether the endoprosthesis is already situated in the position (and/or orientation) as provided by the planning data. If not, further positioning information and, if appropriate, warning indications can be provided (for example required position changes, displacement, rotation, etc.).

It should expressly be pointed out at this juncture that the first image data set and the second image data set need not necessarily be acquired by the same modality, rather that different modalities (for example x-ray and magnetic resonance imaging) can also be used here. In this case, additional registration measures are used in order to be able to correlate the two image data sets.

Preferably, a 3D/2D registration is carried out in order to convert the preoperative three-dimensional image data set into the intraoperatively acquired two-dimensional image data set, and vice versa. In this case, recourse can be had to known registration procedures from the prior art. In this respect, reference is made, for example, to the publication “Automatic Localization of Vertebral Levels in X-ray Fluoroscopy Using 3D-2D Registration: A Tool to Reduce Wrong-Site Surgery”, Y. Otake, S. Schafer, J. W. Stayman, W. Zbijewski, G. Kleinszig, R. Graumann, A. J. Khanna and J. H. Siewerdsen, 2012, published in Phys. Med. Biol. 57(17): 5485-5508 (2012).

A major advantage of the control function according to the invention can be seen in the fact that the image data sets (preoperative and intraoperative) that are acquired anyway are used to derive planning data for the endoprosthesis and thus to be able to provide relevant control measures without the patient having to be confronted with an additional radiation loading. In one preferred embodiment, in each case one preoperative and one intraoperative recording are used. However, it likewise lies within the scope of the invention also to acquire a plurality of first image data sets and second image data sets and to use them for the further image processing for the generation of control data. The first and second image data sets can be two- and/or three-dimensional image data sets. If a two-dimensional data set is acquired preoperatively and a three-dimensional data set is acquired intraoperatively, 2D-3D registration measures are correspondingly employed.

In accordance with one preferred embodiment, the planning data are implemented using a bone model generated from the first image data set and/or using a prosthesis model. The bone model is patient-specific and represents the respective anatomical conditions (for example femur, generally without the head of the femur for the shaft of the endoprosthesis and acetabulum for the socket of the endoprosthesis). In general (patient-specific endoprostheses also exist) the prosthesis model is not patient-specific, but rather prosthesis-specific and can be provided as a 3D model by the manufacturer of the respective implant. This usually involves a grid model available in a standard format (e.g. STL).

If no prosthesis model can be provided, in an alternative embodiment of the invention it is also possible to generate the prosthesis model. Additional image recordings of the prosthesis may be necessary for this purpose.

In contrast to the previous procedures for inserting endoprostheses, control data can advantageously be provided by the proposal according to the invention, the control data supporting the physician during the insertion and during the placement of the implant components. In particular, the physician is informed of the extent to which the current position corresponds to the planning data. In the event of lack of correspondence, deviations between DESIRED and ACTUAL states are visualized (preferably on a monitor). In other words, the operating surgeon obtains immediate and direct feedback for the optimum placement of shaft and socket of a hip endoprosthesis. That proves to be particularly helpful in applications in which a robot-controlled method is used (e.g. with a milling robot or a positioning robot). The invention makes it possible to check the positioning of the robot visually with the aid of the planning data.

In accordance with one aspect, the first image data set with the planning data and the second image data set with the provisionally inserted prosthesis are superimposed. The respective data sets are matched in order to calculate there from a deviation between the current implant location with respect to the planning location.

In one advantageous development of the invention, provision can be made for the choice of the implant parts likewise to be automated. For this purpose, recourse is had to the first image data set (and thus to the respective anatomical structure of the patient) and to the acquired planning data. Parameters for the implant or implants can thereupon be derived with the aid of an additional calculation unit. By way of example, from the first image data set and the planning data it is possible to derive the fact that the patient here is a young person, and so a relatively short shaft length and a small circumference should be chosen for the respective implant. The calculation unit can then produce a proposal for the selection of implant components (for example with regard to size, shape, material composition, etc.).

In one advantageous development, the calculation unit can comprise a monitoring unit. The monitoring unit serves to check the “mutual suitableness” of the selected implant parts. By way of example, the shape and size of the joint shaft in an endoprosthesis has to be geared toward the respective joint head. The monitoring module then serves to check whether the suitable socket has been chosen for the shaft respectively chosen, and vice versa.

In one preferred development, (besides the first image data set), even further medically relevant data sets are taken into account for calculating the planning data. The data sets can comprise, for example, metadata with regard to the patient (age, sex, constitution, etc.), bone composition (e.g. osteoporosis) and/or biomechanical tissue data, etc. The medically relevant data sets can, for example, also relate to the bone shape, bone size, bone density and further material characteristic values (for example elasticity) of the bone.

The type of anchoring of the implant in the bone is advantageously not stipulated. A cemented or a cement-free anchoring of the endoprosthesis in the bone can thus be involved.

As already mentioned above, the preferred embodiment relates to an endoprosthesis for a hip joint. Alternatively, the control can also be applied to other types of prosthesis, such as, for example, for the knee joint, shoulder joint, elbow joint, foot joint or for vertebrae of the vertebral column or for dental implants.

The first and/or second image data set are/is preferably recorded by an x-ray device, in particular by a method of computed tomography. Alternatively, other modalities can also be employed here, such as, for example, ultrasound methods, methods of nuclear spin tomography, etc.

The above-described embodiments of the method according to the invention can also be embodied as a computer program product containing a computer program, wherein the computer is caused to carry out the above-described method according to the invention if the computer program is executed on the computer or on a processor of the computer.

An alternative solution to the problem also consists in a computer program containing computer program code for performing all method steps of the claimed or above-described method if the computer program is executed on the computer. In this case, the computer program can also be stored on a machine-readable storage medium.

An alternative solution to the problem provides a storage medium which is intended for storing the above-described computer-implemented method and is readable by a computer.

It lies within the scope of the invention that not all the steps of the method need necessarily be performed on one and the same computer entity, rather they can also be performed on different computer entities—and thus in a distributed manner. Furthermore, it is possible that individual sections of the above-described method can be performed in one saleable unit and the remaining components in another saleable unit—as it were as a distributed system. Moreover, the sequence of the method steps can be varied, if appropriate.

Other features which are considered as characteristic for the invention are set forth in the appended claims.

Although the invention is illustrated and described herein as embodied in a control system, method and computer program for positioning an endoprosthesis, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a schematic overview illustration of a computer-aided control system with further components, containing various imaging devices; and

FIG. 2 is a flow chart for a control method for positioning an endoprosthesis in accordance with one preferred embodiment of the invention.

### DETAILED DESCRIPTION OF THE INVENTION

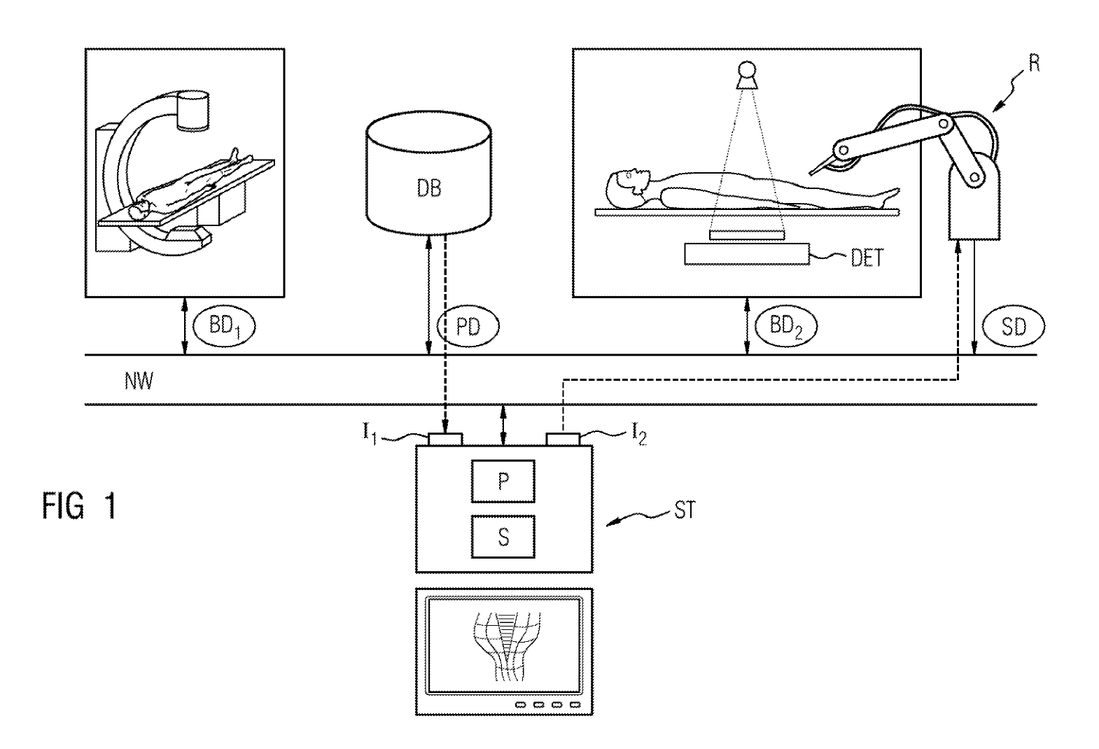

Referring now to the figures of the drawings in detail and first, particularly to FIG. 1 thereof, there is shown a computer-aided medical-technical control system ST. The control system ST serves for controlling a positioning process in the context of an implantation. In the preferred embodiment, the invention relates to hip endoprosthetics and the exact positioning thereof in the body. However, it likewise lies within the scope of the invention to use the control system ST for other implants and/or prostheses to be inserted, such as, for example, for other joints or vessels and in dental technology.

It has been possible for a few decades to implement a total endoprosthetic replacement of, for example, the hip joint. In this case, it is of fundamental importance for the respective implant to be perfectly positioned in the bone and anchored there. For as far as possible permanent durability of the prosthesis in the bone it is essential to take account of various factors and parameters here in order to position the implant in the bone in such a way that the growth of the bone onto the surface of the prosthesis is fostered, in order to obtain the highest possible stability. In principle, the relevance of the correct positioning is independent of the type of respective anchoring, that is to say whether a cement-free or cemented anchoring is chosen.

In the previous procedure it is customary to produce a recording (usually an x-ray recording) preoperatively and, on the basis thereof, to determine the respective implant (with regard to size and shape, inter alia) and to position it in the bone after a resection of the bone, in particular of the neck of the femur, has been performed.

It proves to be particularly disadvantageous here that (for the physician) further indications and data as to how and where the implant is intended to be positioned (if appropriate by a robot) are not available during the operation. The physician relies completely on his knowledge gained from experience during the insertion. This has the consequence that previous instances of planning according to the prior art are only in the nature of approximations and optimum positioning which can be monitored in a computer-aided manner is not possible. This proves to be disadvantageous particularly if part of the tissue has changed pathologically or arthrotically and inaccuracies thus occur during the implementation.

This forms the starting point for the invention, which proposes a computer-aided control system ST with which data about the positioning of the endoprosthesis can be provided even during the operation and thus in real time. In particular, the intention is to provide information about whether the endoprosthesis is situated at the DESIRED position determined in a preceding planning phase and/or, if this is not the case, the intention is to output control data which make it possible (e.g. for the robot) to move from the ACTUAL position to the DESIRED position for the endoprosthesis.

In principle, the invention is not restricted to a physician manually inserting the respective endoprosthesis. A large area of application involves robot-aided implantations, in which recourse is had to one or more robots for implementing the prosthetics. Since the mid-1990s, in Germany, computer-aided shaft implantations have been carried out, in which, for example, a milling robot correspondingly prepares the femur such that the latter can receive the implant. The milling robot is controlled by corresponding control data. Besides the milling robot, an operation robot can also be used, which is controlled via a robot control unit and is used for positioning the implant and for further processes that are to be performed intraoperatively (for example anchoring the implant). According to the invention, all robot-based means (milling robot, operation robot, etc.) are fed with control data of the control system ST.

As illustrated in FIG. 1, the control system ST contains a read-in interface I₁ for acquiring or reading in a first image data set BD₁. The first image data set BD₁ usually originates from a preoperatively acquired CT (computed tomography). However, it is likewise conceivable to take account of other modalities for image acquisition (for example image acquisition by magnetic resonance imaging).

Furthermore, the control system ST contains a processor P for generating planning data PD. The planning data PD can be forwarded to further computer-aided entities; by way of example, they can be visualized on a monitor M. In an initial application, the planning data PD can also be forwarded without further processing by the control system ST to at least one robot R for the control thereof. The planning data PD relate to the intended or planned positioning and orientation of the endoprosthesis in an (anatomical) structure. One preferred embodiment of the invention provides for the generated planning data PD to be integrated into the first image data set BD₁ and thus to identify a DESIRED state for the endoprosthesis. Alternatively, the planning data PD are provided separately with respect to the first image data set BD₁.

The control system ST furthermore contains an intraoperative image interface I₂, which is intended to acquire a second image data set BD₂. The provisionally inserted endoprosthesis is represented visually in the second image data set. In other words, the second image data set BD₂ identifies the respective current ACTUAL state of the endoprosthesis (that is to say where and how the endoprosthesis is positioned in the body).

Furthermore, the control system ST contains a control unit S, which is intended to generate control data SD for the insertion of the endoprosthesis. The control data SD are preferably implemented on the basis of a calculation and/or an image processing. In particular, the DESIRED state and the ACTUAL state of the respective implant are matched in this case. For this purpose, it is possible to have recourse to a 3D/2D registration such as is known in the prior art.

In the preferred embodiment of the invention, however, the control data SD rather than the planning data PD are transmitted to the robot R by the control system ST. The control data contain—in contrast to the planning data PD—the matching with the respective dynamically and currently acquired position of the implant (part).

As illustrated in FIG. 1, in one preferred development of the invention, the control system ST contains a monitor M, on which the processed data sets can be displayed, in particular the first image data set BD₁, the second image data set BD₂ and/or the control data set SD or the planning data PD. It proves to be particularly helpful that a pictorial representation of the comparison between DESIRED and ACTUAL states can also be visualized on the surface of the monitor M. The operating surgeon thus obtains in real time information about whether it is necessary for the surgeon still to finely adjust and position the implant or whether the implant is already situated at the optimum position.

As already mentioned above, the sequence according to the invention is based on recording, besides the preoperatively acquired first image data set BD₁, in addition also at least one further, intraoperatively acquired second image data set BD₂. The second image data set BD₂ identifies the provisionally inserted endoprosthesis in the respective position.

If a deviation between the DESIRED state and the ACTUAL state is evident, then corresponding control data SD are generated and output (optionally optically and/or acoustically, for example via a corresponding acoustic warning indication) and/or the control data SD can be forwarded to a robot-aided device R, for example in order to finely position the implant again.

Consequently, the control data SD are control data for a robot R or for robot-aided devices which are employed in the context of the implantation.

As illustrated in FIG. 1, the control system usually accesses at least one database DB. The processed data sets (first and second image data sets BD₁, BD₂, planning data PD and control data SD) can be stored in the database. Furthermore, it is possible to provide model data here. The model data can contain reference data identifying in what position and in what orientation the endoprosthesis is usually positioned. Furthermore, the model data can contain a grid model of the respective implant. The data are usually provided by the implant manufacturer as a 3D grid model.

During the intraoperative imaging, an x-ray-based imaging is usually used, for example a two-dimensional AP-fluoroscopic recording (ap: anterior-posterior). It is possible, however, also to employ other intraoperative image acquisition methods here.

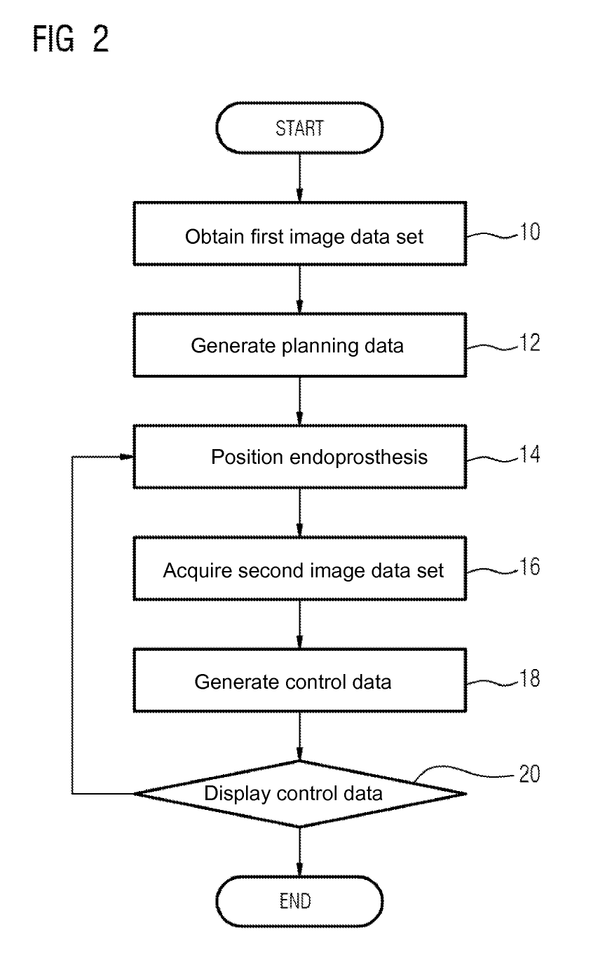

A customary sequence of the process according to the invention is explained in greater detail below with reference to FIG. 2.

After the start of the method, in step 10, the first image data set BD₁ is provided. In this case, the first image data set BD₁ can be acquired directly or it is possible to read out the image data set from recordings already performed previously, from a memory.

In step 12, planning data PD are generated. The planning data PD identify the DESIRED position or the DESIRED state of the implant and, in particular, its position and orientation in the respective anatomical structure (for example femur).

In step 14, the respective endoprosthesis is positioned provisionally (automatically, in a robot-controlled manner or manually). This step presupposes, of course, that an endoprosthesis corresponding accordingly to the stipulations of the planning data PD has been chosen. In other words, the planning data PD also serve for selecting the respective prosthesis.

In step 16, at least the second image data set BD₂ is acquired or provided (for example read in from a memory). What is essential in this case, however, is that the second image data set BD₂ is acquired intraoperatively and represents the respective current provisional position of the endoprosthesis.

In step 18, control data SD are generated, which can serve for controlling a robot R for the fine positioning of the endoprosthesis in the anatomical structure.

In step 20, the generated control data SD are displayed in a visual representation on the monitor M. Furthermore, it is possible for the control data SD to be transmitted to further devices and means, such as, for example, to an operation robot R intended for freely positioning the prosthesis.

Afterward, the method can end. Alternatively, it is possible to perform the method or parts thereof repeatedly or sequentially. This is represented in FIG. 2 by the fact that the method branches again to step 14 in order to bring about a renewed fine positioning of the endoprosthesis.

It goes without saying that a plurality of passes for the fine positioning of the endoprosthesis can also be performed here. The current location is displayed on the monitor M in the ACTUAL state in each case in real time. One preferred development of the invention provides for further implant-relevant parameters and data sets to be provided and derived from data sources, for example the database DB. They involve, for example, the type of bone, bone size, bone density and/or elasticity values or further physical and/or medical properties. Furthermore, data with regard to the respective implant, such as, for example, physical properties of the implant, material composition, size and/or other characteristic values, can be provided in the database DB.

These data sets are then processed by the processor P and/or by the control unit S. In particular, they are employed for generating the control data SD.

One advantageous development provides for applying the method sequentially in order to achieve a fine positioning of the implant in a plurality of steps. In this case, even further intraoperatively acquired recordings (inter alia also from other modalities) can also be acquired and used for calculation by the control system ST. In other words, after each positioning process, dynamically and in each case currently new control data SD are calculated and can be forwarded to the robot R.

Furthermore, the control data SD can also be used for image-aided navigation during the operation and for navigation. “Navigation” can relate to the positioning of the implant and/or to the positioning of operation instruments and further surgical elements (forceps, etc.).

Furthermore, in one preferred development of the invention, the control data SD are passed to further units and/or robots which are used for various operative purposes (as already mentioned above for example for milling out the implant bearing in the femur). Furthermore, the control system ST can be fed with sensor data forwarded as feedback from the robot R to the control system ST. Here it is possible, for example, to carry out processing if the milling robot R moves outside the milling boundaries provided, in order to generate a corresponding warning signal.

Finally, it should be pointed out that the description of the invention and the exemplary embodiments, in principle, should not be understood in a restrictive manner with regard to a specific physical realization of the invention. In particular, it is obvious to a person skilled in the art that the invention can be realized partly or completely in software and/or hardware and/or in a manner distributed among a plurality of physical products—in this case in particular including computer program products.

The following is a summary list of reference numerals and the corresponding structure used in the above description of the invention.

### LIST OF REFERENCE SIGNS

BD₁ first image data set (preoperative)
BD₂ second image data set (intraoperative)
DB database
R robot
SD control data
ST control system
M monitor
NW network
DET detector
P processor
PD planning data
S control unit
I₁ read-in interface
I₂ intraoperative image interface
10 providing the first image data set
12 generating planning data
14 provisionally positioning the endoprosthesis
16 providing or acquiring at least one second image data set in which the provisionally positioned endoprosthesis is visible
18 generating control data for the fine positioning of the endoprosthesis
20 displaying the control data in a visual representation on a monitor M and/or forwarding the control data SD to robots R or further entities

1. A computer-aided control system for use during image-aided insertion of an endoprosthesis, comprising:
a read-in interface for acquiring or reading in at least one first image data set;
a processor for generating planning data and embedding the planning data into the first image data set as a DESIRED state for the endoprosthesis;
an intraoperative image interface for acquiring or reading in at least one second image data set with a provisionally inserted endoprosthesis as an ACTUAL state; and
a control unit for generating control data for the endoprosthesis on a basis of an image-processing comparison between the DESIRED state and the ACTUAL state using a 3D/2D registration.

2. The control system according to claim 1, further comprising at least one monitor on which the first image data set, the second image data set and/or a control data set are displayed, wherein the control data set contains a pictorial representation of the comparison between the DESIRED state and the ACTUAL state.

3. A medical-technical method for image-aided control of an implantation of an endoprosthesis, which comprises the following steps of:
providing at least one preoperatively acquired first image data set;
generating planning data and embedding the planning data into the first image data set as a DESIRED state for the endoprosthesis;
provisionally inserting the endoprosthesis;
acquiring at least one second image data set with the provisionally inserted endoprosthesis as an ACTUAL state; and
generating control data for the endoprosthesis on a basis of an image-processing comparison between the DESIRED state and the ACTUAL state using a 3D/2D registration.

4. The method according to claim 3, which further comprises performing the generating of the planning data using a bone model generated from the first image data set and/or using a prosthesis model.

5. The method according to claim 2, wherein the first image data set and/or the second image data set is a two-dimensional medical image data set or a three-dimensional medical image data set.

6. The method according to claim 1, wherein the first image data set is a CT or MRI image data set.

7. The method according to claim 1, wherein the second image data set is an image data set acquired by means of a C-arc or a fluoroscopic image data set.

8. A computer program product stored in a non-transitory memory of a computer and containing computer-readable instructions for performing a medical-technical method for image-aided control of an implantation of an endoprosthesis, which comprises the following steps of:
providing at least one preoperatively acquired first image data set;
generating planning data and embedding the planning data into the first image data set as a DESIRED state for the endoprosthesis;
provisionally inserting the endoprosthesis;
acquiring at least one second image data set with the provisionally inserted endoprosthesis as an ACTUAL state; and
generating control data for the endoprosthesis on a basis of an image-processing comparison between the DESIRED state and the ACTUAL state using a 3D/2D registration.
